(19) United States
(12) Patent Application Publication
(10) Pub. No.: US 2009/0229028 A1
(43) Pub. Date: Sep. 17, 2009

(54) Article of manufacture for a hat and eye shield and process for making same

(76) Inventor: Selwyn Dobkins, San Diego, CA (US)

Correspondence Address: HARRY V. MCGAHEY JR., 1532 SIXTH AVENUE, SAN DIEGO, CA, 92101, US

(21) Appl. No.: 12/075,637

(22) Filed: Mar. 13, 2008

(51) Int. Cl. A61F 9/00; A42B 1/24; G02C 13/00

(52) U.S. Cl. 2/10; 2/209.13; 351/178

(57) ABSTRACT

An article of manufacture for a hat and eye shield comprised of a hat, a bill attached to the front of the hat having a plurality of vertical openings extending vertically through the distal edge of the bill furthest from the hat, a lens with a plurality of pins extending vertically up from its top surface, the pins on the lens inserted into the vertical openings through the distal edge of the bill furthest from the hat, and the pins on the lens anchored to the vertical openings in the bill by an extrusion means.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of eye protection and more specifically to an article of manufacture for a hat and eye shield and process for making same. Many devices and processes for protecting the eyes of active wearers have been patented. This invention is a way to screen the eyes from the sun in all sports while still keeping the ability to wipe the face and eyes.

Relevant art includes the following:

U.S. Pat. No. 1,298,636 (Altman 1919) discloses a hinged pair of sunglasses anchored to the brim of a hat.

U.S. Pat. No. 1,182,734 (Baker 1916) discloses a flat eyeshade/eye shield which can clip onto the brim of a hat and which can then be raised or lowered. The eyeshade is in a strictly linear design and does not wrap around any portion of the cap to be able to protect the peripheral vision of the wearer.

U.S. Pat. No. 1,514,111 Sutton (1924) discloses a way for a pair of spectacles to be raised and lowered on the brim of a hat. Again there is no provision for protection of the wearer's peripheral vision. US patent application number 2007/0192933 also discloses a method of supporting a pair of spectacles from the brim of a hat.

U.S. Pat. No. 4,951,316 Moody (1990) is an improved sun visor which is comprised of a removable eye shield that can shade the forehead face and eyes in which the eye shield is attached in various adjustable positions and therefore less susceptible to slippage. A snap and loctite member is used to insert a translucent eye shield at varying distances from the wearer's eyes.

U.S. Pat. No. 5,422,686 Kelman (1995) discloses a detachable eyeglass assembly for a hat which includes a clip for removable attachment to the visor.

U.S. Pat. No. 5,778,448 Maher (1998) also discloses another method of assembling and attaching sunglasses to a cap, as does U.S. Pat. No. 6,244,706 (Maher 2001)

Other patents which have detachable sunglass subassemblies include U.S. Pat. No. 5,471,259. Cahill (1995). This patent describes a clip-on assembly for mounting sunglasses to the brim of a hat. A clip secures the assembly to the hat brim and holds the sunglasses, which can also be tilted up toward the bill out of the wearer's line of vision.

U.S. Pat. No. 5,208,916 Kelman (1993), and U.S. Pat. No. 2,481,960 Wall (1949).

U.S. Pat. No. 6,173,447 Arnold (2001) discloses an eye shield that wraps around the brim of a hat and extends downward to protect the wearer's face. The shield is connected to the hat with two clamps that attach at opposite sides of the bill. The shield can also be lifted upward to a non-operative position.

U.S. Pat. No. 5,208,916 Kelman (1993) and U.S. Pat. No. 5,422,686 Kelman (1995) discloses an assembly for mounting sunglasses to the bottom of the bill of a hat. The assembly includes a pair of parallel rails that allow the sunglasses to be adjusted toward or away from the wearer. The sunglasses can be tilted up out of the wearer's line of vision.

Some hats with eye shields have been patented for use in a particular trade. U.S. Pat. No. 4,850,049 Landis et al. (1989) teaches a hat with a protective shield for use in surgery. Safety hats with shields are also disclosed in U.S. Pat No. 3,685,054 Raschke (1972) and U.S. Pat. No. 2,834,017 Simpson et al. (1958).

Other detachable eye shield assemblies are disclosed in U.S. Pat. No. 5,689,827 Ryder (1997), U.S. Pat. No. 4,951,316 Moody (1990), U.S. Pat. No. 6,739,718 Jung (2004) and U.S. Pat. No. 1,514,111 Sutton (1924). Each has a unique way of attaching the eye shield to a hat.

U.S. Pat. No. 2,481,960 Wall et al. (1949), and U.S. Pat. No. 6,662,371 Shin (2003) as well as application Nos. 2007/0079422 Wu (2007) and 2007/0192933 Kam (2007) disclose other hat and eye shield combinations.

None of these inventions utilize a method of protecting both the front and peripheral vision of an active wearer engaged in vigorous physical activities in such a manner that the shields will not be subject to movement or falling off. In this invention the shield always remains fixed in its location while providing the user both sun protection and an unobstructed view under the lens of the area in front of the wearer. This is a particularly desirable quality when a person is active in an ocean or other similar environment where an unobstructed view is preferred.

One problem that exists especially with water sports where sun glasses are important, is that sunglasses block a person's ability to wipe the water from their face. In addition sunglasses become smudged, often by sun block and this further blocks one's vision while participating in sports. In addition, when participating in cold water sports, sunglasses fog quickly and often render themselves useless almost immediately. Another problem that this invention will solve is for those who need to wear prescription lens eyeglasses. They will be able to continue to wear their prescription eyeglasses without having to buy expensive darkening lenses or other apparatus.

This invention changes the current paradigm of eye protection when wearing sunglasses by removing them from close contact with the face. Instead the eye protection is at the edge brim of the hat. In the preferred embodiment between 2.5 and 5 inches away from the eyes.

The subject invention involves both a novel process of construction during manufacture as well as of design. This invention will benefit most outdoor activities from baseball games to hunting. It will especially benefit those who participate in active water sports.

### SUMMARY OF THE INVENTION

The primary object of the invention is an improved type of eye shield and method of manufacturer thereof.

Another object of the invention is to place the eye shield at the end of the hat's brim rather than the typical resting of it on the wearer's nose. In addition, the wearer has the ability to easily wipe his or her face and eyes during use.

Another object of the invention is to allow active participation in sports, especially water sports. The invention will be particularly useful to surfers, but also to any others with exposure to bright sunlight.

Other objects and advantages of the present invention will become apparent from the following descriptions, taken in connection with the accompanying drawings, wherein, by way of illustration and example, an embodiment of the present invention is disclosed. For instance the hat could be a skull cap or other type of covering for the head. The hat bill could also be a visor. The lens could be a prescription lens and it could be any shade as well as an ordinary transparent lens.

In accordance with a preferred embodiment of the invention, there is disclosed an article of manufacture for a hat and eye shield comprising: a hat, a bill attached to the front of the hat having a plurality of vertical openings extending vertically through the distal edge of the bill furthest from the hat, a lens with a plurality of pins extending vertically up from its top surface, the pins on the lens inserted into the vertical openings through the distal edge of the bill furthest from the hat, and the pins on the lens anchored to the vertical openings in the bill by an extrusion means which squashes or melts the pins down so as to form a rivet.

In accordance with another preferred embodiment of the invention, there is disclosed a method for constructing a hat and eye shield comprising the steps of: attaching a bill to the front of a hat, the bill having a plurality of vertical openings extending vertically through the distal edge of the bill furthest from the hat, inserting a lens with a plurality of pins extending vertically up from its top surface into the vertical openings through the distal edge of the bill furthest from the hat, and anchoring the pins on the lens to the vertical openings in the bill by an extrusion means.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is an elevational view of the invention.

FIG. 2 is a plan view of the invention.

FIG. 3 is a perspective view of the invention.

FIG. 4 is a perspective view of the invention.

FIG. 5 is an elevational view of the invention.

FIG. 6 is a perspective view of the invention.

FIG. 7 is an elevational view of the invention.

FIG. 8 is a perspective view of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

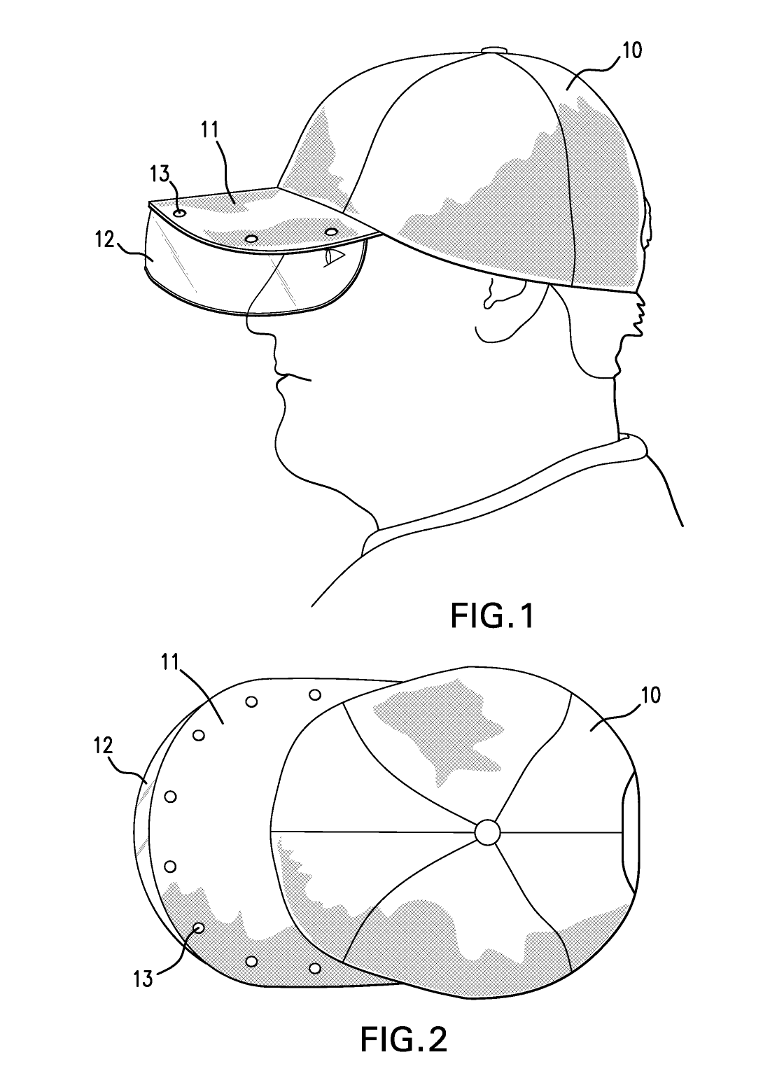

In accordance with the present invention, FIG. 1 is an elevational view of the invention which is an article of manufacture for an eye shield. The hat (10) is seen on the wearer connected to the bill (11). The extrusion squashed or melted down studs or rivets (13) of the vertical stationary pins anchoring the lens (12) to the bill (11) comprising a unique method of attachment can also be seen.

FIG. 2 is a plan view of the invention from above. The hat (10) connected to the bill (11) with the studs (13) anchoring the stationary pins of the lens (12) are all visible [The stationary pins (19) are shown in FIG. 6]. These studs (13) are attached to the bill (11) at its distal most edge the furthest from the front of the hat by having a plurality of openings extending vertically through the bill (11) at the place where the studs (13) are inserted.

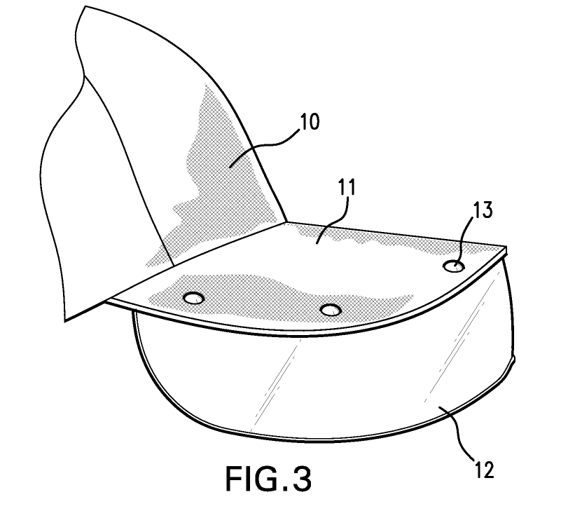

FIG. 3 is a perspective view of the invention showing the lens (12) anchored by the studs (13) to the bill (11) of the hat (10).

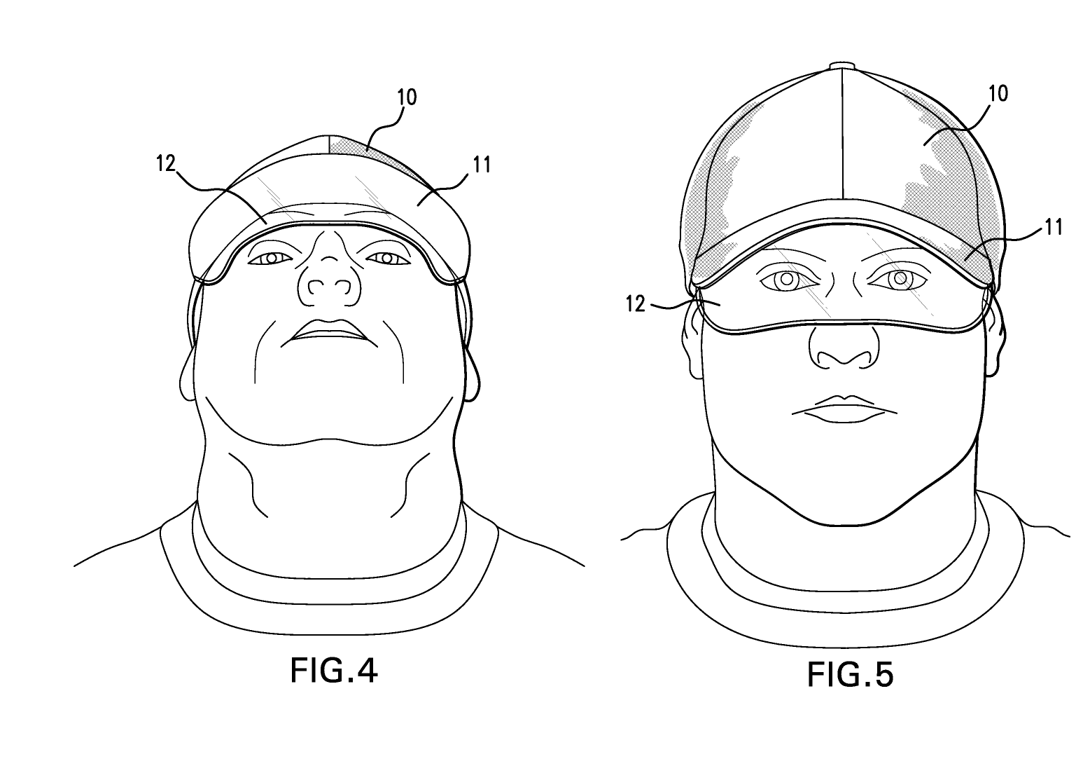

FIG. 4 is a perspective view of the invention showing the wearer able to lift his or her head so as to see under the lens (12) if desired.

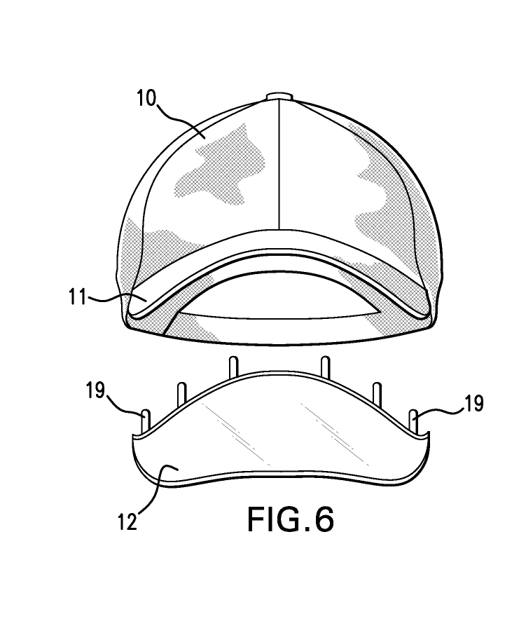

FIG. 5 is an elevational view of the invention showing the lens protecting the eyes when the wearer lowers his or her head.

FIG. 6 is a perspective view of the invention from the front showing the stationary pins (19) which are anchored to the lens and about to be inserted into the bill (11). These pins (19) are anchored into the bill of the hat by one of the steps of the manufacturing process by using an extrusion process which expands the pins (19) at the upper top surface of the bill (11) after the pins (19) have been inserted up through the top surface of the bill (11).

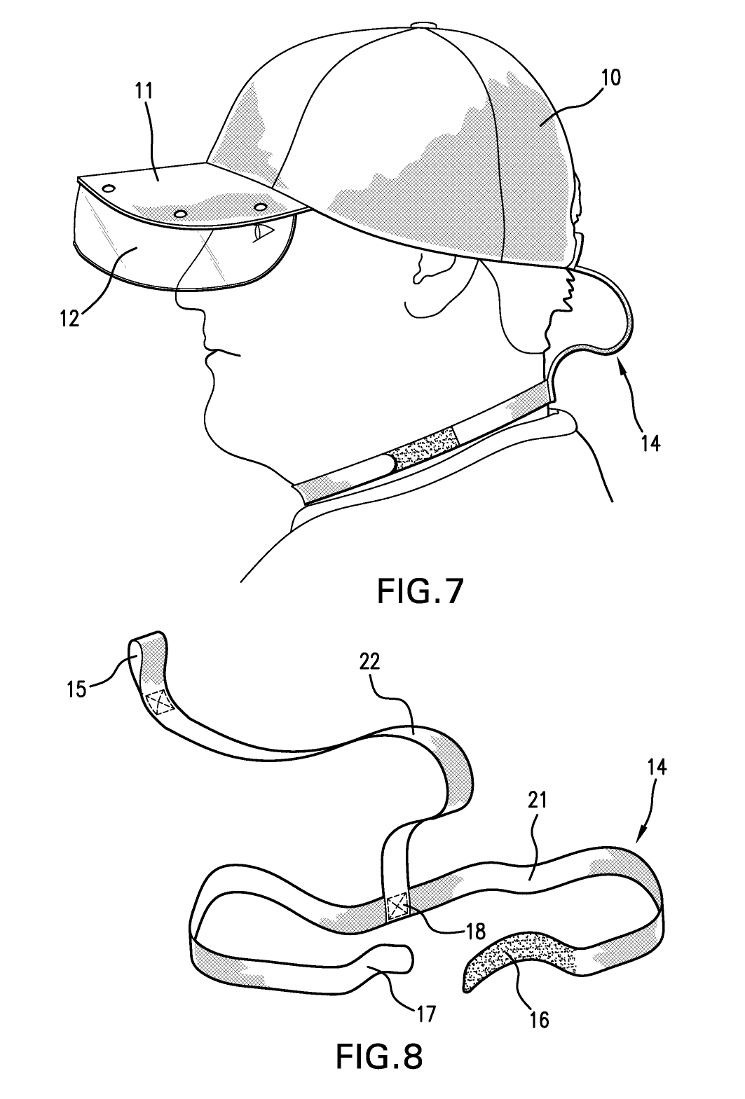

FIG. 7 is an elevational view of the strap portion of the invention (14) showing how the anchor strap assembly (14) is used to keep the hat from being lost during vigorous activities.

FIG. 8 is a perspective view of the leash strap portion of the invention (14) to keep the wearer from losing the hat showing the anchor means (15) which in the preferred embodiment is comprised of a leash loop connected to a hat strap (22) to anchor the hat strap (22) to the hat, the anchor point comprised of a leash strap (18) to anchor the strap to the collar using a neck tie (21), and the attachment means comprised of a female Velcro (17) and a male Velcro (18) to anchor the collar neck tie (21) to the leash strap (22).

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. An article of manufacture for a hat and eye shield comprising:
a hat;
a bill attached to the front of the hat having a plurality of vertical openings extending vertically through the bill near the distal edge of the bill furthest from the hat;
a lens with a plurality of stationary pins extending vertically up from its top surface;
the pins on the lens inserted into the vertical openings through the bill; and
the pins on the lens anchored to the vertical openings in the bill by an extrusion means.

2. The hat and eye shield in claim 1 wherein the vertical openings extending vertically through the bill extend through the distal edge of the bill furthest from the hat.

3. The hat and eye shield in claim 1 wherein the lens is positioned exactly on the distal edge of the bill furthest from the hat.

4. The hat and eye shield in claim 1 wherein the lens is placed on the bill so that it is between 2.5 and 5 inches away from the wearer's eyes.

5. The hat and eye shield in claim 1 wherein the lens is a prescription lens fitted to the eye prescription of the wearer.

6. The hat and eye shield in claim 5 wherein the lens is also a shaded lens.

7. The hat and eye shield in claim 5 wherein the lens is also a colored lens.

8. The hat and eye shield in claim 1 wherein the lens is transparent.

9. The hat and eye shield in claim 1 wherein the lens is colored.

10. The hat and eye shield in claim 1 wherein the pins on the lens are anchored to the vertical openings in the bill by an extrusion means.

11. The hat and eye shield in claim 1 wherein the pins on the lens are anchored to the vertical openings in the bill by an extrusion means which melts the pins down so as to form a rivet.

12. The hat and eye shield in claim 1 wherein the pins on the lens are anchored to the vertical openings in the bill by an extrusion means which squashes the pins down so as to form a rivet.

13. The hat and eye shield in claim 1 wherein it contains an anchor means to keep the hat from being lost comprised of:
a leash loop;
the leash loop connected to a hat strap;
the hat strap anchored to a leash strap;
the leash strap anchored to the wearer by a collar using a collar neck tie;
the collar neck tie secured by an attachment means.

14. The hat and eye shield in claim 13 wherein:
the attachment means for the collar neck tie is comprised of a female Velcro and a male Velcro attachment means.

15. A process for manufacturing a hat and eye shield comprising the steps of:
attaching a bill to the front of a hat, the bill having a plurality of vertical openings extending vertically through the bill near the distal edge of the bill furthest from the hat;
inserting a lens with a plurality of stationary pins extending vertically up from its top surface into the vertical openings through the bill;
and anchoring the pins on the lens to the vertical openings in the bill by an extrusion means.

16. The process in claim 15 wherein the vertical openings extending vertically through the bill extend through the distal edge of the bill furthest from the hat.

17. The process in claim 15 wherein the lens is positioned exactly on the distal edge of the bill furthest from the hat.

18. The process in claim 15 wherein the lens is placed on the bill so that it is between 2.5 and 5 inches away from the wearer's eyes.

19. The process of claim 15 wherein the pins on the lens are anchored to the vertical openings in the bill by an extrusion means which melts the pins down so as to form a rivet.
